(19) United States
(12) Patent Application Publication
(10) Pub. No.: US 2025/0339643 A1
(43) Pub. Date: Nov. 6, 2025

(54) MEDICAL DEVICES AND ASSEMBLIES FOR DELIVERING FLUID AND RELATED METHODS OF MANUFACTURE

(71) Applicant: Boston Scientific Scimed, Inc., Maple Grove, MN (US)

(72) Inventors: Kevin L. BAGLEY, Natick, MA (US); Nathan STENGER, Minneapolis, MN (US); Justin Theodore NELSON, Vadnais Heights, MN (US); Deocraciano Aristocrascio CARDOSO, Marlborough, MA (US)

(73) Assignee: Boston Scientific Scimed, Inc., Maple Grove, MN (US)

(21) Appl. No.: 19/196,279

(22) Filed: May 1, 2025

Related U.S. Application Data
(60) Provisional application No. 63/642,251, filed on May 3, 2024.

(51) Int. Cl. A61M 25/00; A61M 39/10; A61M 39/24

(52) U.S. Cl. CPC A61M 25/0068; A61M 25/001; A61M 25/0045; A61M 25/0052; A61M 39/10; A61M 39/24; A61M 2025/0004; A61M 2039/1027; A61M 2205/0216; A61M 2205/0238; A61M 2207/00

(57) ABSTRACT

A medical device may include a sheath having a proximal end, a distal end, and a lumen extending between the proximal end and the distal end. The medical device may include a plurality of strands fixed to and extending distally from the distal end of the sheath. Each strand of the plurality of strands may include a proximal fixed end and a distal free end. The plurality of strands may surround a distal opening of the sheath.

### CROSS-REFERENCE TO RELATED APPLICATIONS

This application claims the benefit of priority to U.S. Provisional Application No. 63/642,251, filed on May 3, 2024, which is incorporated by reference herein in its entirety.

### TECHNICAL FIELD

Various aspects of this disclosure relate generally to medical devices and assemblies for delivering one or more fluids and related methods of manufacturing thereof. More specifically, aspects of the disclosure pertain to devices, assemblies, and/or methods of manufacturing thereof for delivering one or more fluids to a target site, via medical devices, such as endoscopes.

### BACKGROUND

Bleeding ulcers or other wound sites may occur, for example, in a subject's gastrointestinal (GI) tract. For example, following another diagnostic or treatment procedure, such as endoscopic mucosal resection (EMR) or endoscopic submucosal dissection (ESD), bleeding may need to be prevented or treated. In another example, a line of sutures in a GI tract may be in need of reinforcement. In a further example, a fistula may require treatment. In yet another example, peroral endoscopic myotomy (POEM) sites may need to be closed. Therefore, a need exists for devices and assemblies to deliver agents for treating or preventing bleeding.

### SUMMARY

The disclosure includes devices and assemblies, for delivering one or more fluids to a target site of a subject, for example, to help heal an ulcer and/or to perform hemostasis, and related methods of manufacturing thereof. Each of the aspects disclosed herein may include one or more of the features described in connection with any of the other disclosed aspects.

According an example, a medical device may include a sheath having a proximal end, a distal end, and a lumen extending between the proximal end and the distal end. The medical device may include a plurality of strands fixed to and extending distally from the distal end of the sheath. Each strand of the plurality of strands may include a proximal fixed end and a distal free end. The plurality of strands may surround a distal opening of the sheath.

Any of the devices disclosed herein may include any of the following features, additionally or alternatively, in any combination.

Each free end of the plurality of strands may be distal to the distal opening of the lumen. The free end of each strand of the plurality of strands may be flexible. Each strand of the plurality of strands may be integrally formed with the sheath. Each strand of the plurality of strands may have a same length. A durometer of each strand of the plurality of strands may be less than a durometer of the sheath. Each strand of the plurality of strands may have a hydrophilic or hydrophobic coating. The hydrophilic or hydrophobic coating may be disposed on a radially inner surface of each strand, a radially outer surface of each strand, or both.

A proximal portion of the sheath may include a supporting member. The supporting member may be a heat shrink or a coiled member. The proximal end of the sheath may include a connector in fluid communication with the lumen of the sheath. The connector may be one of a luer lock connector, a quick connect fitting, or a one-way valve. The medical device may be configured to deliver a fluid through the lumen of the sheath. The lumen of the sheath may be a first lumen, and the sheath may be configured to be movably disposed within a second lumen. The second lumen may be a lumen of a scope or a catheter.

In another example, a medical device may include a sheath having a proximal end, a distal end, and a lumen extending between the proximal end and the distal end. The medical device may include a plurality of bristles fixed to and extending radially outward from a surface of the distal end of the sheath.

Any of the devices disclosed herein may include any of the following features, additionally or alternatively, in any combination.

The plurality of bristles may be arranged in a series of columns or rows along the surface of the distal end of the sheath. The sheath may include one or more openings disposed between two or more of the columns or rows of the plurality of bristles.

Aspects of this disclosure includes methods of manufacturing a distal end of the sheath. The method may include cutting a wall of the sheath to create one or more strands, heating the one or more strands, and pulling the one or more strands distally relative to the sheath.

Any of the methods disclosed herein may include any of the following steps, additionally or alternatively, in any combination.

The method may further include cooling the one or more strands, and cutting a distal end of the one or more strands. The cuts may be perpendicular to a longitudinal axis of the sheath.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1A is a side view of an exemplary medical device, according to aspects of this disclosure.

FIG. 1B depicts a distal portion of the medical device shown in FIG. 1A, according to aspects of this disclosure.

FIG. 2 depicts a flow chart of an exemplary method of manufacturing the medical device shown in FIGS. 1A and 1B, according to aspects of this disclosure.

FIG. 3 depicts a flow chart of another exemplary method of manufacturing the medical device shown in FIGS. 1A and 1B, according to aspects of this disclosure.

FIGS. 4-9 illustrate a distal portions of alternative medical devices, according to aspects of this disclosure.

FIGS. 10A and 10B illustrate cross sectional views of a distal portion of a further alternative medical device in a first configuration (FIG. 10A) and in a second configuration (FIG. 10B).

### DETAILED DESCRIPTION

A fluid may be used to prevent or treat bleeding or other conditions in a GI tract. In examples, a fluid may be delivered to the target site(s) to form a protective layer that helps to treat or minimize delayed bleeds, potential perforations, and/or stricture formations. Once the fluid is delivered to the target site(s) within the subject, the material may be difficult to apply or spread around the target site(s) using conventional devices. For example, applying or spreading the fluid around the target site using conventional devices may be difficult and/or time-consuming for the user. Furthermore, applying or spreading the fluid around the target site using rigid or semi-rigid conventional devices may injure the subject's tissue.

Aspects of this disclosure seek to improve and/or ease a user's ability to deliver a fluid to a target site. In aspects, a medical device may be inserted via a natural orifice or incision or via a working channel of an insertion device, such as a medical scope (e.g., endoscope). Various aspects of this disclosure may help the user perform wound treatment and/or hemostasis within the subject, reduce overall procedure time, reduce overall procedure costs, etc. Each of the embodiments of this disclosure is configured to apply and/or distribute a fluid around the target site.

While the disclosure primarily relates to highly viscous fluids on the order of 200 centipoise or greater, one of ordinary skill in the art may recognize that the disclosure may be applicable to fluids of any viscosity. Examples of fluids (e.g., biocompatible viscous fluids) that may be applied to a target site using the devices disclosed herein may include, but are not limited to, fibrin, chitosan, thrombin, fluids including calcium salts, cyanoacrylates, albumin and glutaraldehyde, poly (ethylene glycol) (PEG), polyurethane, etc. In aspects, such fluids may be endoscopically delivered adhesives or other agents that help to create a protective layer that minimizes potential perforations, delayed bleeds, and/or stricture formations.

One of ordinary skill in the art will appreciate that the devices and assemblies of this disclosure may be used with a variety of biocompatible fluids and that the devices and assemblies of the disclosure may be applicable to various medical procedures beyond bleeding control.

Reference will now be made in detail to examples of the present disclosure described above and illustrated in the accompanying drawings. Wherever possible, the same reference numbers will be used throughout the drawings to refer to the same or like parts. Where feasible, reference numbers ending in the same tens and ones digit (the same last two digits) refer to analogous elements. Throughout various figures, “P” and “D” arrows may be used to illustrate a proximal or distal direction, respectively.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value.

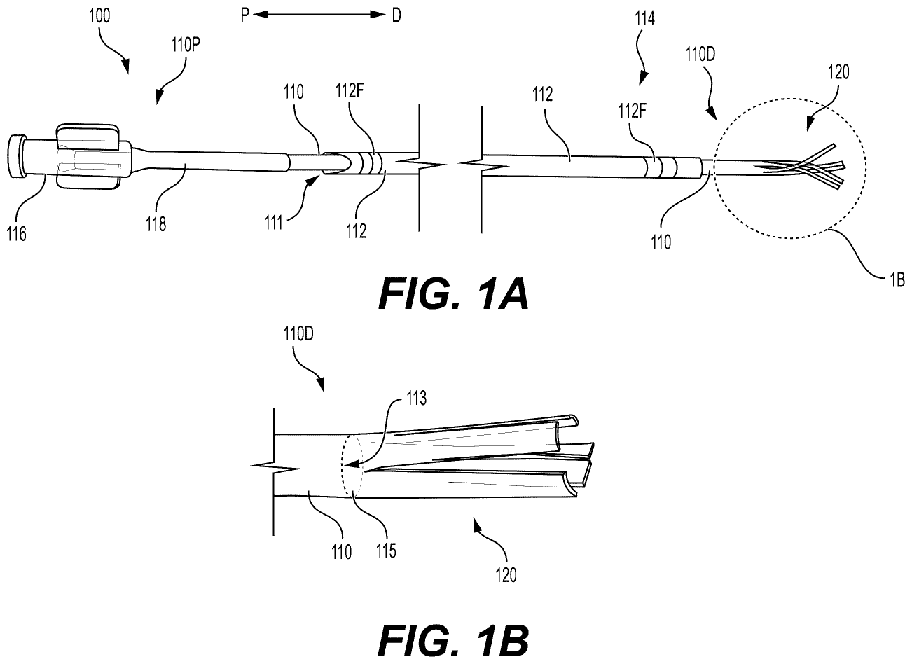

FIG. 1A depicts a side view of an exemplary medical device 100. Medical device 100 may be used to deliver and apply a fluid. As shown in FIG. 1A, medical device 100 may feature an inner sheath 110 disposed within a lumen 111 of an outer sheath 112. Together, inner sheath 110 and outer sheath 112 may comprise a sheath assembly 114 of medical device 100. Inner sheath 110 may be movable relative to outer sheath 112 (e.g., within lumen 111 of outer sheath 112). For example, inner sheath 110 may be moved proximally and/or distally relative to outer sheath 112 and/or inner sheath 110 may be rotated about a longitudinal axis of inner sheath 110 and relative to outer sheath 112. A proximal end 110P of inner sheath 110 may include a connector 116 that is in fluid communication with a lumen 113 (FIG. 1B) of inner sheath 110. Lumen 113 may extend between proximal end 110P and a distal end 110D of inner sheath 110. Distal end 110D of inner sheath 110 may include a variety of features discussed throughout this disclosure to assist in the delivery and application of a fluid to a target site. For example, as shown in FIGS. 1A and 1B, distal end 110D of inner sheath 110 may include a plurality of strands 120, discussed in more detail below. FIG. 1B provides a close-up view of strands 120 of distal end 110D.

Referring to FIG. 1A, connector 116 may be any connector commonly used in the art to fluidly connect two components. Connector 116 may include, for example, a luer lock connector, a quick connect fitting, a one-way valve, etc. A luer lock connector is shown in FIG. 1A. Connector 116 may be configured to permit the connection of a fluid source, such as a syringe, to inner sheath 110. In these aspects, fluid may be delivered through an opening 115 (FIG. 1B) at distal end 110D of inner sheath 110, for example, via lumen 113 of inner sheath 110.

Although not shown, inner sheath 110 may include two or more lumens, with each lumen having its own connector. In these aspects, two or more fluids may be delivered to the target site. The two or more fluids may be the same or they may be different from one another. For example, a multi-part fluid may be delivered to the target site via a multi-lumen inner sheath 110. In these aspects, a first fluid source containing a first fluid part (Part A) may be fixed to a first connector that is in fluid communication with a first lumen of inner sheath 110. A second fluid source containing a second fluid part (Part B) may be fixed to a second connector that is in fluid communication with a second lumen of inner sheath 110. Together or separately, each of part A and part B may injected through their respective lumens of inner sheath 110 and delivered to the target site. Once each part comes in contact with the other (e.g., after being delivered from inner sheath 110), the parts may crosslink. The cross-linked structure of the resulting combination of parts A and B may have enhanced hemostatic properties compared to parts A or B individually. In a non-limiting example, part A may be fibrinogen (e.g., lyophilized pooled human concentrate) and part B may be thrombin (e.g., of bovine or human origin). The biocompatible fibrinogen and thrombin mixture may also contain calcium salts.

Referring still to FIG. 1A, connector 116 may be fixedly coupled (directly or indirectly) to proximal end 110P of inner sheath 110 using an adhesive, a swage, a mechanical fastener, and/or any other method or combination of methods to fix two components together. In some aspects, a stiffening member 118 may be applied around an outer surface of proximal end 110P of inner sheath 110. Stiffening member 118 may at least partially overlap and surround a distal end of connector 116. In these aspects, stiffening member 118 may assist in securing connector 116 to inner sheath 110.

In some aspects, stiffening member 118 may assist in strengthening a portion of inner sheath 110, for example, to prevent proximal end 110P of inner sheath 110 from kinking or bending abruptly during a procedure. Stiffening member 118 may be a heat shrink, a coil, or any other member commonly used in the art to prevent a sheath from kinking or abruptly bending. Additionally or alternatively, inner sheath 110 may be extruded such that proximal end 110P of inner sheath 110 is inherently stiffer or more rigid as compared to more distal portions of inner sheath 110 (e.g., distal end 110D). The stiffer material of proximal end 110P of inner sheath 110 may assist in preventing kinks or abrupt bends.

Sheath assembly 114 (e.g., inner sheath 110 and outer sheath 112) may define a longitudinal axis of medical device 100. Sheath assembly 114 may be comprised of flexible or semi-rigid materials such that, for example, sheath assembly 114 has sufficient flexibility to navigate a subject's tortuous anatomy. For example, sheath assembly 114 may translate proximally and/or distally through a subject's tortuous anatomy, e.g., via a lumen of a scope (e.g., an endoscope, not shown) or other delivery device. In some aspects, medical device 100 may be inserted directly into the subject, for example, via a natural orifice or incision. During the insertion and/or navigation of distal end 110D to a target site of a subject, distal end 110D may be entirely or partially disposed within lumen 111 of outer sheath 112. For example, a distalmost end of outer sheath 112 may be more distal than distal end 110D.

Before, during, or after the distal end 110D of medical device 100 has been navigated to the target site, outer sheath 112 may be pulled proximally relative to inner sheath 110 and/or inner sheath 110 may be pushed distally relative to outer sheath 112 such that at least a portion of distal end 110D extends beyond a distalmost end of outer sheath 112. During removal and/or repositioning of medical device 100 within the subject, inner sheath 110 and/or outer sheath 112 may be moved in opposite directions such that distal end 110D of inner sheath 110 is disposed at least partially within lumen 111 of outer sheath 112. In these aspects, outer sheath 112 may assist in preventing any fluids/materials that are stuck to or otherwise remaining on distal end 110D from being inadvertently applied to other tissue or obstructing the lumen of the delivery device, for example, when inner sheath 110 is removed from the subject. In these aspects, outer sheath 112 may assist with the delivery and/or removal of inner sheath 110 within the subject.

Outer sheath 112 may include a variety of features 112F (e.g., markings, protrusions, indentations, etc.) disposed on an outer surface of one or more portions of outer sheath 112. For example, features 112F on a distal or proximal portion of outer sheath 112 may include graduated markings to assist in providing the user with a visual scale. Features 112F may additionally or alternatively include one or more indentations and/or protrusions on the proximal portion of outer sheath 112, for example, to assist a user with gripping outer sheath 110. Features 112F may be integrally formed with and/or fixed to outer sheath 112.

Shown in detail in FIG. 1B, distal end 110D of inner sheath 110 may include one or more strands 120, or bristles. Strands 120 may be strips of material. Strands 120 may be integrally formed with inner sheath 110. Alternatively, strands 120 may be formed separately and fixed to distal end 110D of inner sheath 110. A distal opening of the lumen of inner sheath 110 may be adjacent to and/or formed by a fixed end of each strand 120. For example, each strand 120 may have a fixed proximalmost end that is fixed to distal end 110D of inner sheath 110 or integrally formed therewith. In these aspects, the distal opening of the lumen of inner sheath 110 may be proximal relative to the distalmost end of each strand 120. Strands 120 may be formed with or attached to an entire circumference of inner sheath 110. In other aspects, strands 120 may be formed with or attached to just a portion of a circumference of inner sheath 110. In these aspects, strands 120 may not extend around an entirety of inner sheath 110.

Each strand 120 may also have a free, distalmost end that is not attached or otherwise fixed to inner sheath 110. In some aspects, strands 120 may resemble the bristles of a paintbrush. In aspects, each strand 120 may be pliant (e.g., flexible or bendable). In aspects, each strand 120 may be approximately 1.0 millimeter to over 25.0 millimeters (e.g., 5 millimeters to 20 millimeters) in length. A desired length of strands 120 may be based on the type of fluid that is being delivered and/or the size of the target site. For example, strands 120 that are longer may assist with spreading the fluid on/around a larger target site. In other aspects, strands 120 that are shorter may be desired to prevent the fluid from being spread too far past the target site. The desired length of strands 120 may also be chosen based on the fluid being delivered. For example, a more viscous fluid may require shorter strands 120 and/or a less viscous fluid may require longer strands 120.

Each strand 120 may have one or more of the same dimensions relative to one another. For example, each strand 120 may have a same longitudinal length (e.g., measured from the fixed end to the free end of each strand 120), a same width (e.g., measured perpendicular to a longitudinal axis of each strand 120 and a diameter/radius of inner sheath 110), and/or a same thickness (e.g., measured from a radially inward-facing surface to a radially outward-facing surface of each strand 120). In other aspects, strands 120 may have one or more varying dimensions.

In a nominal (neutral) position of inner sheath 110, e.g., when strands 120 are not disposed within lumen 111 of outer sheath 112, one or more strands 120 may splay radially outward. For example, a perpendicular distance measured from the free, distalmost end of a strand 120 to the longitudinal axis of inner sheath 110 may be greater than a perpendicular distance as measured from the fixed end of the same strand 120 to the longitudinal axis of inner sheath 110. Additionally or alternatively, in a nominal position, one or more strands 120 may be parallel to the longitudinal axis of inner sheath 110, and/or one or more strands 120 may extend radially inward. In some aspects, strands 120 extend in varying directions.

One or more strands 120 may be formed of and/or coated with a hydrophobic and/or a hydrophilic material. The material or coating of strands 120 may, in some aspects, assist in preventing the fluid delivered via inner sheath 110 from sticking to strands 120. For example, a hydrophobic coating may prevent the fluid from adhering to the surface of each strand 120. In other examples, a hydrophilic coating may assist in delivering the fluid from inner sheath, for example, such that the fluid moves distally, e.g., via capillary action, along the surface of each strand 120. In some aspects, each strand 120 may be formed of or coated with two or more materials. For example, a proximal portion of each strand 120 may be formed of or coated with a first hydrophilic or hydrophobic material, and a distal portion of each strand 120 may be formed of or coated with a second hydrophobic or hydrophilic material.

Additionally or alternatively, an outward-facing surface of each strand 120 may be formed of, or coated, with a first hydrophilic or hydrophobic material and an inward-facing surface of each strand 120 may be formed of, or coated with, a second hydrophilic or hydrophobic material. In some aspects, the first and second materials may both each be hydrophobic or hydrophilic materials. In other aspects, the first material may be hydrophobic and the second material may be hydrophilic, or vice versa. Additionally or alternatively, one or more strands 120 may be formed of, or coated, with a material that activates the fluid during delivery or application, as discussed above.

During use, the user may insert at least a portion of medical device 100 through a lumen of a delivery device (e.g., scope or other catheter) and a distal portion of medical device 100 to a target site within the subject. Before or after reaching the target site, distal end 110D of inner sheath 110 may be extended distally from outer sheath 112 (via relative proximal movement of outer sheath 112 or relative distal movement of inner sheath 110, as discussed above), e.g., such that strands 120 are disposed distally relative to a distalmost end of outer sheath 112. The user may connect a fluid source to connect 116 and inject a fluid through lumen 113 of inner sheath 110. The fluid may then be delivered to a target site (e.g., via opening 115 of lumen 113 of inner sheath 110).

During or after the fluid has been delivered, the user may use strands 120 to spread, apply, or otherwise distribute the material on and around the target site. For example, the user may manipulate the delivery device to “paint” the fluid on/around the target site. Additionally or alternatively, medical device 100 and/or inner sheath 110 may be manipulated (e.g., translated proximally/distally within the delivery device or subject) to “paint” the fluid on/around the target site. In these aspects, strands 120 may resemble the bristles of a paintbrush and assist in spreading, applying, or otherwise distributing the fluid on/around the target site. In aspects, the compliant nature of each strand 120 may reduce a risk causing harm to the tissue or the subject.

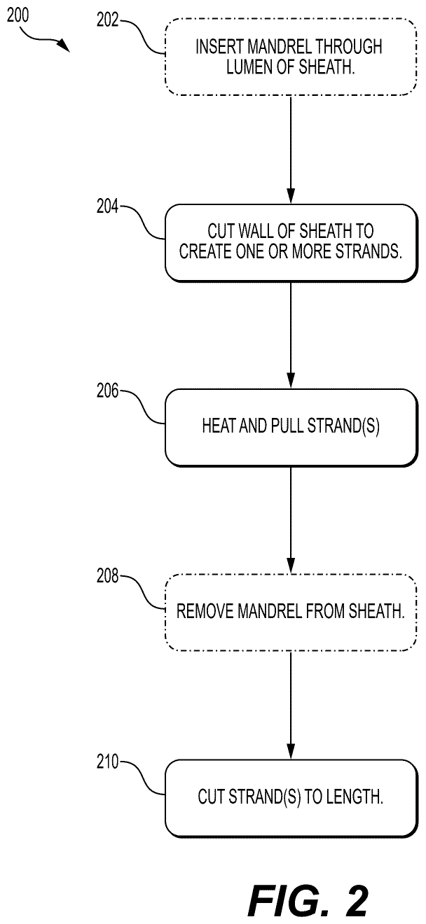

FIG. 2 illustrates an exemplary method 200 that may be used to manufacture strands 120 of FIGS. 1A and 1B. For example, in a first, optional step 202, a mandrel may be inserted through a lumen of a sheath, or catheter, such as inner sheath 110. The mandrel may assist in preventing the inner diameter of the sheath from shrinking during the following steps.

In a next step 204, a wall of the sheath may be cut proximally from a distal end of the sheath one or more times. The cuts may be longitudinal, e.g., parallel to a longitudinal axis of the sheath and/or parallel to one another. In some aspects, the mandrel mentioned above with respect to step 202 may have one or more features that assist in cutting the sheath longitudinally. For example, the mandrel may be formed with one or more sharp blades that extend radially outward. As such, when the mandrel is inserted through the lumen of the sheath, the wall of the sheath may be cut. In other aspects, a user may use a blade or other cutting tool to form the longitudinal cuts within the wall of the sheath. The cuts may be any desired length. Strands, similar to strands 120 shown in FIGS. 1A and 1B, may be formed between the cuts.

In a following step 206, one or more of the strands formed by the cuts may be heated and pulled distally. The heating and pulling processes may be done simultaneously or consecutively. Heating the strand(s) may begin to melt the material and make each strand more pliant, or easier to pull distally. As the strand(s) are pulled distally, a longitudinal length of each strand may increase. Furthermore, as the material of each strand cools, a shape and length of each strand may be fixed. The mandrel, optionally inserted through the lumen of the sheath in step 202, may assist in preventing the inner diameter of the sheath from being reduced, particularly as the strand(s) is/are heated and pulled distally. In aspects, the process of heating and pulling the strands distally may result in the strands being more flexible or bendable relative to proximal portions of the sheath.

If a mandrel is used, it may be removed from the lumen of the sheath in a step 208.

In a final step 210, the strand(s) may be cut to a desired length. For example, one or more strands may be cut perpendicularly to the longitudinal axis of the sheath. The resulting sheath may resemble inner sheath 110 and distal end 110D of FIGS. 1A and 1B.

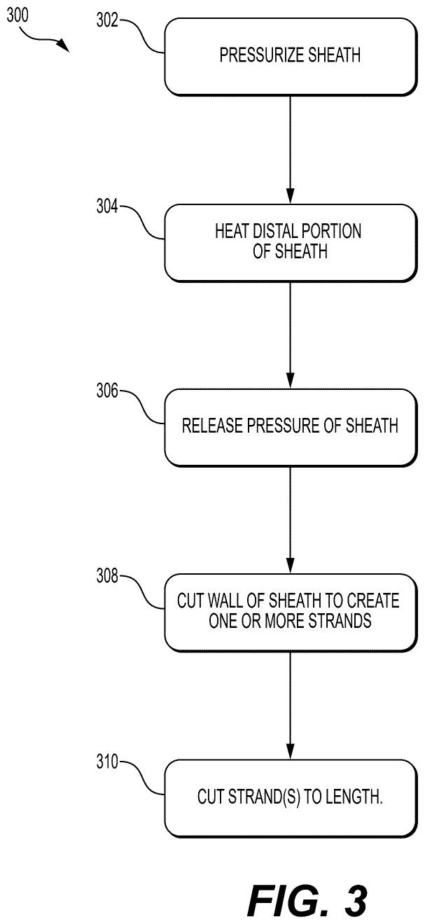

FIG. 3 illustrates another exemplary method 300 that may be used to manufacture strands 120 of FIGS. 1A and 1B. For example, in a first step 302, a lumen of a sheath (e.g., inner sheath 110) may be pressurized. For example, a positive pressure source may be attached to a first end (e.g., proximal or distal end) of the sheath and be in fluid communication with the lumen of the sheath. The opposite end (e.g., distal or proximal end) of the sheath may be sealed or closed.

With the sheath pressurized, in a next step 304, heat may be applied to a distal portion of the sheath. The heat may increase a temperature of the material forming the distal portion of the sheath, making it more pliant. Because positive pressure is being applied to the lumen of the sheath, the portion being heated may expand to form a balloon-like structure.

Once the balloon-like structure reaches a desired dimension (e.g., diameter and/or length), the sheath may be removed from the heat source and the pressure within the sheath may be released in a step 306.

In a step 308, the walls forming the newly-formed balloon of the sheath may be cut to create one or more strands. For example, longitudinal cuts (e.g., cuts that are parallel to a longitudinal axis of the sheath) may be created within the balloon. A distal end of the balloon may be cut off (e.g., perpendicular to the longitudinal axis of the sheath). With the distal end of the balloon removed, strands similar to strands 120 of FIGS. 1A and 1B may be formed between the longitudinal cuts.

In a final step 310, the strands may be cut to length. The resulting sheath may resemble inner sheath 110 and distal end 110D of FIGS. 1A and 1B.

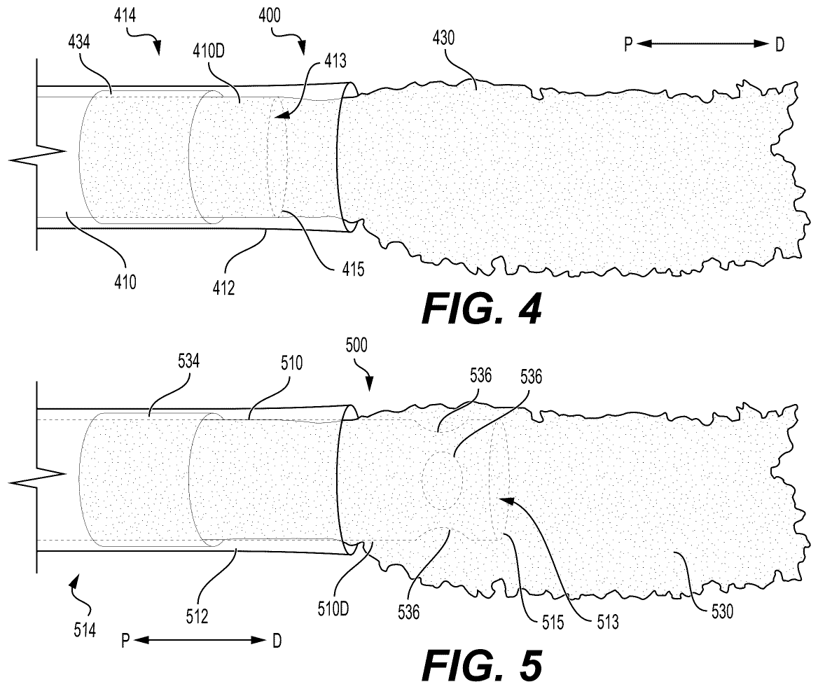

FIG. 4 illustrates a distal portion of another medical device 400. Medical device 400 may have any or all of the same characteristics of medical device 100 of FIGS. 1A and 1B, except as described below. For example, medical device 400 may include a sheath assembly 414 comprised of inner sheath 410 and outer sheath 412. Inner sheath 410 may be moveable relative to outer sheath 412. Although not shown, a proximal end of each of inner sheath 410 and outer sheath 412 may have any or all of the same characteristics as the proximal end of inner sheath 110 and outer sheath 112 of FIG. 1A, respectively. For example, the proximal end of inner sheath 410 may include a connector to fluidly connect a fluid source to inner sheath 410. A porous body 430 may be fixed to a distal end 410D of inner sheath 410.

Sheath assembly 414 of FIG. 4 is shown in an actuated (extended) stated (e.g., with at least a portion of porous body 430 extended distally past a distalmost end of outer sheath 412). In some aspects, a distalmost end of inner sheath 410 may be proximal of a distalmost end of outer sheath 412 in the actuated stated, and porous body 430 may be distal of the distalmost end of outer sheath 412. In other aspects, the distalmost end of inner sheath 410 may be adjacent to or distal of the distalmost end of outer sheath 412, and porous body 430 may be distal of the distalmost end of outer sheath 412. In an unactuated (retracted) stated, a distalmost end of each of inner sheath 410 and a distalmost end of porous body 430 may be proximal of the distalmost end of outer sheath 412.

In aspects, a proximal end of porous body 430 may at least partially overlap with distal end 410D of inner sheath 410. For example, porous body 430 may be fixed to and at least partially surround an outer surface of distal end 410D of inner sheath 410. Aspects of inner sheath 410 are shown in broken lines in FIG. 4, for example, to illustrate that distal end 410D of inner sheath 410 may extend through a portion of porous body 430. Porous body 430 may be fixed to inner sheath 410 via a fastener 434. Fastener 434 may include a crimp, an adhesive or epoxy, a swage, a heat shrink, etc. Fastener 434 may be proximal of a distalmost end of inner sheath 410.

Porous body 430 may be a sponge or open-cell foam. For example, porous body 430 may be flexible, compressible, porous, sterile, and/or disposable. Suitable materials include polyurethanes, esters, ethers, composite materials, and any medical-grade material. In aspects, porous body 430 may include therapeutic additives to aide in the delivery of a fluid via inner sheath 410 and/or to otherwise treat a subject. In the actuated (extended) state of sheath assembly 414, porous body 430 may expand such that a diameter of porous body 430 is greater than a diameter of inner sheath 410 and/or outer sheath 412. In the unactuated (retracted) state of sheath assembly 414, porous body 430 may be compressed such that the diameter of porous body is less than or equal to an inner diameter of outer sheath 412.

During use, a fluid may be delivered through a lumen 413 of inner sheath 410. As the fluid is injected out of inner sheath (e.g., via a distalmost opening 415 of inner sheath 410), porous body 430 may absorb and/or become saturated with the fluid. The fluid may then be applied to the target site by, for example, “painting” a target site with porous body 430. For example, as porous body 430 contacts the tissue of the target site, the fluid may be transferred onto the tissue from porous body 430. In these aspects, a size of the pores or voids, a density, and/or a flexibility of the material comprising porous body 430 may be selected based on a desired performance of porous body 430. For example, a porous body 430 having a greater density, smaller pores, and/or decreased flexibility may be suitable in applying a fluid of lower viscosity. In other aspects, a porous body 430 having a lower density, larger pores, and/or increased flexibility may be suitable in applying a fluid of higher viscosity.

FIG. 5 illustrates a distal portion of another medical device 500. Medical device 500 may have any or all of the same characteristics of medical device 100 and/or medical device 400, except as described below. For example, medical device 500 may include a sheath assembly 514 comprised of an inner sheath 510 and an outer sheath 512. Although not shown, a proximal end of each of inner sheath 510 and outer sheath 512 may have any or all of the same characteristics as proximal end 110P of inner sheath 110 and a proximal end of outer sheath 112 of FIG. 1A, and/or proximal ends of inner sheath 410 and outer sheath 412 of FIG. 4. For example, the proximal end of inner sheath 510 may include a connector to fluidly connect a fluid source to inner sheath 510. A porous body 530 may be fixed to distal end 510D of inner sheath 510 via a fastener 534. Porous body 530 and fastener 534 may each have any or all of the same characteristics of porous body 430 and fastener 434 of FIG. 4, respectively.

Sheath assembly 514 of FIG. 5 is shown in an actuated (extended) stated. (e.g., with at least a portion of porous body 530 extended distally past a distalmost end of outer sheath 512). In the actuated state, distal end 510D of inner sheath 510 may be adjacent to or distal of a distalmost end out outer sheath 512. In an unactuated (retracted) stated, a distalmost end of each of inner sheath 510 and porous body 530 may be proximal of a distalmost end of outer sheath 512.

Distal end 510D of inner sheath 510 may have a distalmost opening 515 and one or more side openings 536 disposed on a side surface of inner sheath 510, for example, proximal of the distalmost end of inner sheath 510. Porous body 530 may extend over and/or at least partially cover distalmost opening 515 and side openings 536. Openings 515 and/or 536 may be in fluid communication with a lumen 513 of inner sheath 510. The one or more side openings 536 of inner sheath 510 may facilitate the delivery of a fluid into porous body 530. Although three ovular, or circular, side openings 536 are shown in FIG. 5, additional side openings of any shape or size may be disposed around the side surface of inner sheath 510. For example, additional side openings 536 may be proximal of side openings 536 shown in FIG. 5.

In some aspects, distalmost opening 515 of inner sheath 510 may be closed or shut off. In these aspects, the fluid may only flow into porous body 530 via side opening(s) 536.

During use, a fluid may be delivered through lumen 513 of inner sheath 510. As the fluid is injected out of inner sheath (e.g., via distalmost opening 515 of inner sheath 510 and/or side opening(s) 536), porous body 530 may absorb and/or become saturated with the fluid. The fluid may then be applied to the target site by, for example, “painting” the target site with porous body 530.

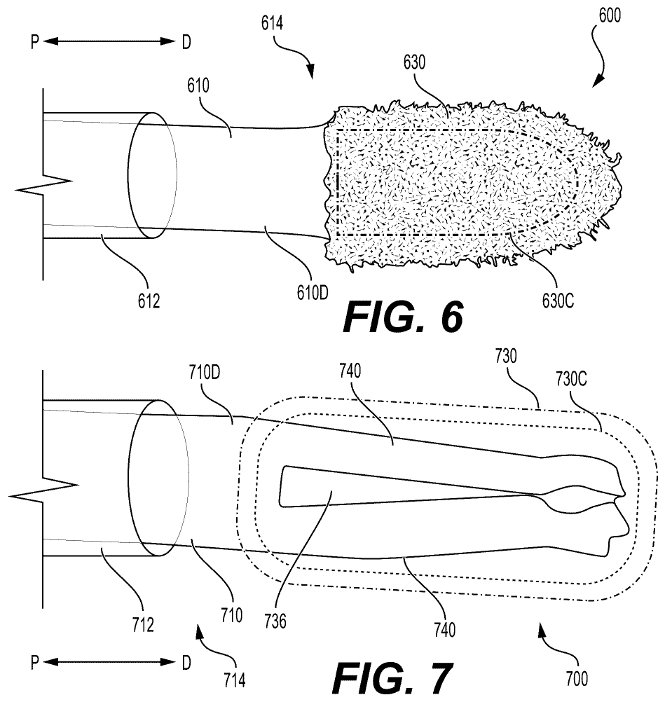

FIG. 6 illustrates a distal portion of another medical device 600. Medical device 600 may have any or all of the same characteristics of any of medical devices 100, 400, and/or 500, discussed above, except as described below. For example, medical device 600 may include a sheath assembly 614 comprised of an inner sheath 610 and an outer sheath 612. Porous body 630 may be fixed to distal end 610D of inner sheath 610 via an adhesive or epoxy, and/or fusing or thermal welding techniques.

Porous body 630 may have any or all of the characteristics of porous bodies 430, 530, discussed above, except as described below. Porous body 630 may have a hollowed center 630C. Porous body 630 may be fixed to inner sheath 610 such that, for example, a fluid from inner sheath 610 flows into hollowed center 630C of porous body 630. The fluid may be absorbed by the walls that surround the hollowed center 630C and form porous body 630. The fluid may then be applied to the target site by, for example, “painting” the target site with porous body 630.

FIG. 7 illustrates a distal portion of another medical device 700. Medical device 700 may have any or all of the same characteristics of any of medical devices 100, 400, 500, and/or 600, discussed above, except as described below. For example, medical device 700 may include a sheath assembly 714 comprised of an inner sheath 710 and an outer sheath 712. A porous body 730 may be fixed to distal end 710D of inner sheath 710. Porous body 730 is shown in broken lines, for example, to illustrate one or more supports 740 extending longitudinally therethrough. Support(s) 740 may provide structural support to porous body 730, while still permitting fluid to flow from inner sheath 710 and into porous body 730. Support(s) 740 may extend at least partially through porous body 730. In aspects, support(s) 740 may extend at least partially into a hollowed center 730C of porous body 730, which may be similar to hollowed center 630C of FIG. 6.

In aspects, supports 740 may be integrally formed with inner sheath 710. In some aspects, support(s) 740 may taper inwards, for example, towards a central longitudinal axis of inner sheath 710. In other aspects, support(s) 740 may extend parallel to the longitudinal axis of inner sheath 710. Openings 736 may be formed between support(s) 740 or in supports 740 themselves. Openings 736 may permit fluid to flow from inner sheath 710 and into, or through, porous body 730. In these aspects, the fluid may be absorbed by porous body 730 and then applied to the target site by, for example, “painting” the target site with porous body 730.

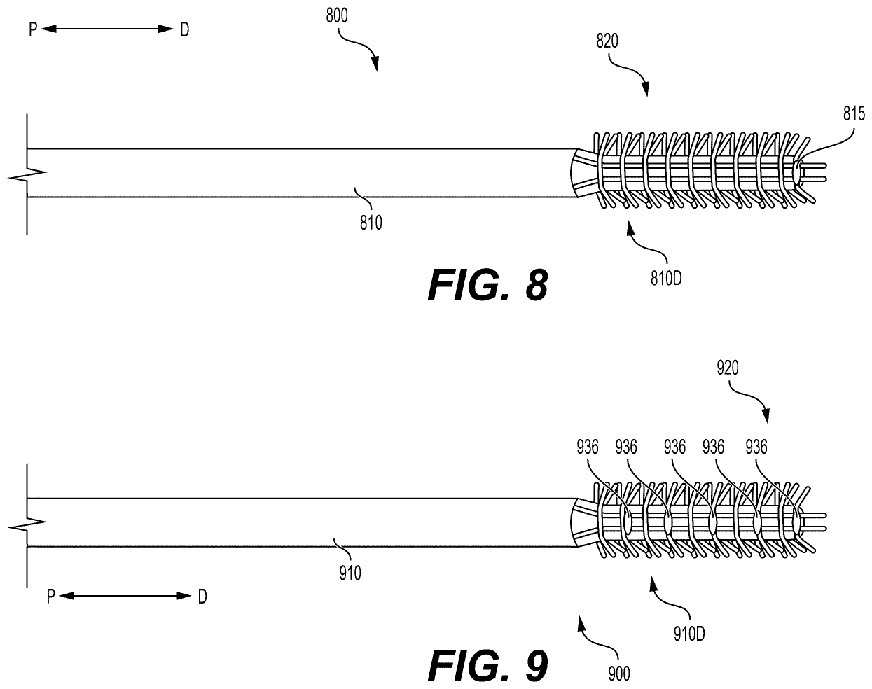

FIG. 8 illustrates a distal portion of another medical device 800. Medical device 800 may have any or all of the same characteristics of any of medical devices 100, 400, 500, 600 and/or 700, discussed above, except as described below. Medical device 800 may include a sheath 810. Although an outer sheath is not shown, sheath 810 may be an inner sheath of a sheath assembly. For example, sheath 810 may be movably disposed within a lumen of an outer sheath (e.g., similar to outer sheath 112, 412, 512, 612, and/or 712). Sheath 810 may have any or all of the same characteristics of inner sheaths 110, 410, 510, 610, and/or 710, except as described below.

A distal end 810D of sheath 810 may include a plurality of bristles 820 extending radially outward from an outer surface of sheath 810. Each of the bristles 820 may be soft and/or flexible. In aspects, each of the bristles 820 may resemble the bristles of a toothbrush or a pipe cleaner. For example, each of the bristles 820 may be fibers or fiber-like elements (e.g., strands) extending from the surface of the sheath 810. Bristles 820 may be made from a plastic, a rubber, and/or a nylon material. Bristles 820 may be arranged on distal end 810D of sheath 810 in a series of columns or rows. The columns or rows may be perpendicular to a longitudinal axis of sheath 810. In other aspects, the columns or rows may additionally or alternatively be parallel to the longitudinal axis of sheath 810. In further aspects, bristles 820 may extend around the outer surface of sheath 810 in a spiral.

A distal opening 815 of sheath 810 may be disposed on a distalmost end of sheath 810. For example, distal opening 815 may be distal to the plurality of bristles 820. Bristles 820 may extend circumferentially around an entirety of the outer surface of sheath 810. In other aspects, bristles 820 may be disposed around a portion of the outer surface of sheath 810 or may be disposed on discrete portions on the outer surface of sheath 810.

In aspects, distal end 810D of sheath 810 may have a same durometer and/or be comprised of a same material as compared to proximal portions of sheath 810. In other aspects, distal end 810D may be comprised of a material having a lower durometer, for example, as compared to more proximal portions of sheath 810. For example, distal end 810D may be more flexible and/or softer as compared to more proximal portions of sheath 810. In these aspects, when distal end 810D comes in contact with the tissue of a target site, distal end 810D may bend or flex.

During use, a fluid may be injected through a lumen of sheath 810 to a target site via distal opening 815. During and/or after delivery of the fluid from sheath 810, the plurality of bristles 820 may be used to aide in the application or apply or distribution of the fluid on or around the target site. For example, bristles 820 may be used to “paint” the fluid on/around the target site.

FIG. 9 illustrates a distal portion of another medical device 900. Medical device 900 may have any or all of the same characteristics of any of medical devices 100, 400, 500, 600, 700, and/or 800, discussed above, except as described below. Medical device 900 may include a sheath 910. Although an outer sheath is not shown, sheath 910 may be the inner sheath of a sheath assembly. For example, sheath 910 may be movably disposed within a lumen of an outer sheath (e.g., similar to outer sheaths 112, 412, 512, 612, and/or 712). Sheath 910 may have any or all of the same characteristics of inner sheaths 110, 410, 510, 610, 710, and/or 810, except as described below.

A distal end 910D of sheath 910 may have any or all of the same characteristics of distal end 810D, discussed above with respect to FIG. 8. For example, a plurality of bristles 920 may be arranged in a series of columns and/or rows and extend radially outward from an outer surface of sheath 910. One or more openings 936 may be disposed between/among the rows or columns of bristles 920. In some aspects, the distalmost end of the sheath 910 may be closed, or without a distalmost opening. In other aspects, the distalmost end of sheath 910 may include a distalmost opening, similar to opening 815 of sheath 810 (FIG. 8).

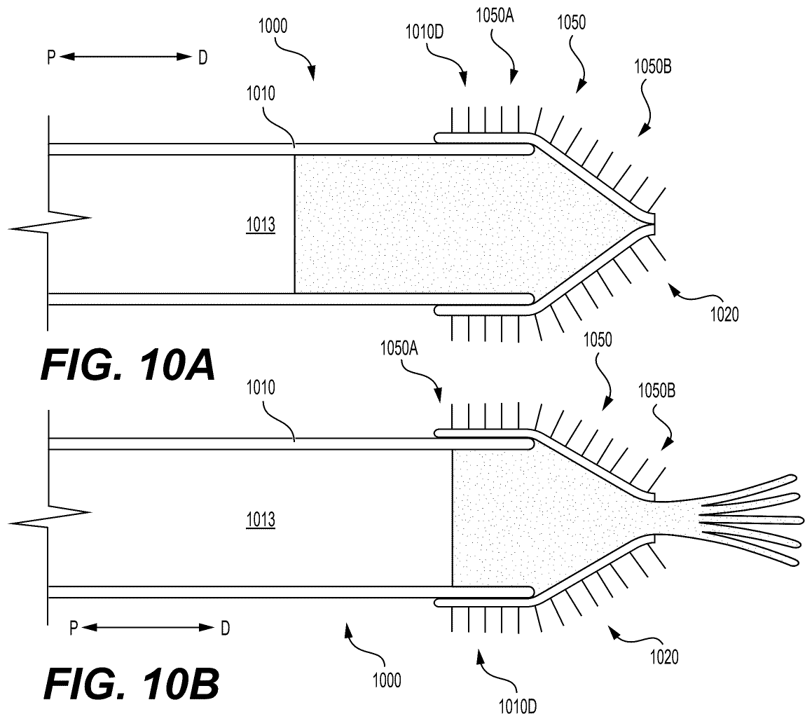

FIGS. 10A and 10B each illustrate a cross section of a distal portion of another medical device 1000 in a first, closed configuration (FIG. 10A) and a second, open configuration (FIG. 10B). Medical device 1000 may have any or all of the characteristics of medical device 100, 400, 500, 600, 700, 800, and/or 900, described above, except as described below. Medical device 1000 may include a sheath 1010. Although an outer sheath not shown, sheath 1010 may be the inner sheath of a sheath assembly. For example, sheath 1010 may be movably disposed within a lumen of an outer sheath (e.g., similar to outer sheaths 112, 412, 512, 612, and/or 712). Sheath 1010 may have any or all of the same characteristics of inner sheaths 110, 410, 510, 610, 710, and/or 810, except as described below.

A distal end 1010D of sheath 1010 may include a valve 1050. Valve 1050 may be a one-way valve. Valve 1050 may have a first, proximal portion 1050A and a second, distal portion 1050B. First portion 1050A may surround a surface of distal end 1010D. For example, first portion 1050A may longitudinally overlap with a portion of distal end 1010D of sheath 1010. First portion 1050A may be secured to distal end 1010D using an adhesive or other chemical fastener. Additionally or alternatively, a fastener (e.g., similar to fastener 434 of FIG. 4 or fastener 534 of FIG. 5) may at least partially surround first portion 1050A of valve 1050 and sheath 1010. The fastener may assist in securing first portion 1050A to sheath 1010.

Second portion 1050B of valve 1050 may taper towards a central longitudinal axis of sheath 1010. Second portion 1050B may extend distally past a distalmost end of sheath 1010. In the first configuration (FIG. 10A), distalmost ends of second portion 1050B may abut one another, thus forming a seal. The seal may prevent fluid from flowing distally out of sheath 1010. In the second configuration (FIG. 10B), distalmost ends of second portion 1050B may be spaced apart, thus forming an opening through which fluid may flow distally. Valve 1050 may transition from the first configuration to the second configuration, for example, by applying a positive pressure on a fluid within sheath 1010. Valve 1050 may transition from the second configuration to the first configuration, for example, by applying a negative pressure and/or by ceasing the application of positive pressure on the fluid within sheath 1010.

Valve 1050 (e.g., first portion 1050A and second portion 1050B) may be comprised of a flexible material (e.g., a rubber, latex, nylon, etc.). In these aspects, when the fluid is delivered from sheath 1010, valve 1050 may transition from the first configuration (FIG. 10A) to the second configuration (FIG. 10B), e.g., by nature of the flexible material of valve 1050. Similarly, when fluid is no longer being delivered, valve 1050 may transition from the second configuration (FIG. 10B) to the first configuration (FIG. 10A).

An outer surface of valve 1050 may include a plurality of bristles 1020 extending outward therefrom. Bristles 1020 may have any or all of the same characteristics of strands 120 and/or bristles 820, and/or 920, discussed above.

During use, medical device 1000 may be navigated to the treatment site in the first configuration (FIG. 10A). Before or after reaching the target site, a positive pressure may be applied to a fluid contained within a lumen 1013 of sheath 1010 to transition valve 1050 to the second configuration (FIG. 10B). For example, the positive pressure may force the distalmost ends of second portion 1050B of valve 1050 to space apart, thus permitting fluid flow out of valve 1050. The positive pressure on the fluid may contained within the lumen 1013 may stop or a negative pressure may be applied to the fluid, thus transitioning valve 1050 back to the first configuration (FIG. 10A). During or after delivery of the fluid to the target site, strands 120 may be used to apply or distribute the fluid on/around the target site. For example, strands 120 may be used to “paint” the fluid on/around the target site.

Each of the aforementioned devices, assemblies, and methods may be used to treat a target site with a medical device. A distal portion of the medical device may include features, for example, to assist in applying and/or distributing a delivered fluid around the target site. By providing a medical device with these features, treatment of a subject (e.g., patient) may be achieved with a high degree of efficiency and precision through the use of one or more medical devices discussed herein.

Furthermore, aspects of each of the devices and assemblies discussed herein may be used in conjunction with one another. For example, a medical device may include strands 120 of medical device 100 and bristles 820 of medical device 800 or bristles 920 of medical device 900. In such an example, a medical device may include strands 120 on a first, distalmost portion of an inner sheath (e.g., inner sheath 110) and bristles 820 and/or 920 may be disposed on a second, proximal portion of the inner sheath. By way of another example, a medical device may include a porous body (e.g., porous body 430, 530, 630, or 730) and a plurality of strands (e.g., strands 120) or bristles (e.g., bristles 820 or 920). Other combinations of features may be contemplated.

It will be apparent to those skilled in the art that various modifications and variations may be made in the disclosed devices, and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
a sheath having a proximal end, a distal end, and a lumen extending between the proximal end and the distal end; and
a plurality of strands fixed to and extending distally from the distal end of the sheath, wherein each strand of the plurality of strands includes a proximal fixed end and a distal free end, and wherein the plurality of strands surrounds a distal opening of the sheath.

2. The medical device of claim 1, wherein each free end of the plurality of strands is distal to the distal opening of the lumen.

3. The medical device of claim 2, wherein the free end of each strand of the plurality of strands is flexible.

4. The medical device of claim 1, wherein each strand of the plurality of strands is integrally formed with the sheath.

5. The medical device of claim 1, wherein each strand of the plurality of strands has a same length.

6. The medical device of claim 1, wherein a durometer of each strand of the plurality of strands is less than a durometer of the sheath.

7. The medical device of claim 1, wherein each strand of the plurality of strands has a hydrophilic or hydrophobic coating.

8. The medical device of claim 7, wherein the hydrophilic or hydrophobic coating is disposed on a radially inner surface of each strand, a radially outer surface of each strand, or both.

9. The medical device of claim 1, wherein a proximal portion of the sheath includes a supporting member.

10. The medical device of claim 9, wherein the supporting member is a heat shrink or a coiled member.

11. The medical device of claim 1, wherein the proximal end of the sheath includes a connector in fluid communication with the lumen of the sheath.

12. The medical device of claim 11, wherein the connector is one of a luer lock connector, a quick connect fitting, or a one-way valve.

13. The medical device of claim 1, wherein the medical device is configured to deliver a fluid through the lumen of the sheath.

14. The medical device of claim 1, wherein the lumen of the sheath is a first lumen, and wherein the sheath is configured to be movably disposed within a second lumen.

15. The medical device of claim 14, wherein the second lumen is a lumen of a scope or a catheter.

16. A medical device, comprising:
a sheath having a proximal end, a distal end, and a lumen extending between the proximal end and the distal end; and
a plurality of bristles fixed to and extending radially outward from a surface of the distal end of the sheath.

17. The medical device of claim 16, wherein the plurality of bristles is arranged in a series of columns or rows along the surface of the distal end of the sheath.

18. The medical device of claim 17, wherein the sheath includes one or more openings disposed between two or more of the columns or rows of the plurality of bristles.

19. A method of manufacturing a distal end of a sheath, comprising:
cutting a wall of the sheath to create one or more strands;
heating the one or more strands; and
pulling the one or more strands distally relative to the sheath.

20. The method of claim 19, further comprising:
cooling the one or more strands; and
cutting a distal end of the one or more strands, wherein the cuts are perpendicular to a longitudinal axis of the sheath.
